(19) United States
(12) Patent Application Publication
(10) Pub. No.: US 2009/0093723 A1
(43) Pub. Date: Apr. 9, 2009

(54) ULTRASOUND DEVICE INCLUDING DISPENSER

(75) Inventor: Adnan I. Merchant, Fremont, CA (US)

Correspondence Address: FULWIDER PATTON - CABOCHON AESTHETICS, 6060 CENTER DRIVE, TENTH FLOOR, LOS ANGELES, CA, 90045, US

(73) Assignee: CABOCHON AESTHETICS, INC., Menlo Park, CA (US)

(21) Appl. No.: 12/245,647

(22) Filed: Oct. 3, 2008

Related U.S. Application Data
(60) Provisional application No. 60/978,058, filed on Oct. 5, 2007.

(51) Int. Cl. A61B 8/14

(52) U.S. Cl. 600/459

(57) ABSTRACT

An ultrasound device, comprising: a housing; an ultrasound transducer disposed within the housing, the transducer having a wear layer; at least one port formed in the housing; a lubricant reservoir operably connected to the at least one port; and a pump in fluid connection with the lubricant reservoir, the pump dispensing lubricant from the reservoir through the at least one port.

### CROSS-REFERENCES TO RELATED APPLICATIONS

This application claims priority from U.S. Provisional Application No. 60/978,058, filed Oct. 5, 2008 incorporated by reference in its entirety.

### FIELD OF THE INVENTION

The present invention relates to an ultrasound device including an integral dispenser for dispensing a lubricant.

### BACKGROUND

Ultrasound devices are typically used in conjunction with an acoustically transparent lubricant, e.g., ultrasound gel, which acoustically couples the device to the treatment area. The ultrasound operator manually applies the gel to the treatment area before actuating the ultrasound device. Unfortunately, ultrasound gel has a tendency to dry over time thereby necessitating periodic reapplication of the ultrasound gel, which disrupts the ultrasound treatment and soils the operator's hands. This also slows down the procedure when multiple placements of the transducer are required. Accordingly, what is needed is an improved way to apply ultrasound gel.

### SUMMARY OF THE INVENTION

Disclosed is an ultrasound device, comprising a housing, an ultrasound transducer disposed within the housing and having a wear layer, at least one lubricant dispensing port formed on one of the housing and the wear layer, a lubricant reservoir operably connected to the at least one lubricant dispensing port, a pump in fluid connection with the lubricant reservoir, the pump dispensing lubricant from the reservoir through the at least one dispensing port, and a pump actuator operably connected to the pump and controlling the pump.

In some embodiments the ultrasound device may include two or more lubricant dispenser ports and a manifold in fluid connection with the pump for distributing lubricant among the ports.

In some embodiments the pump actuator is a foot switch or a hand operated switch provided on the housing.

In some embodiments the at least one dispensing port is formed along a periphery of the wear layer, in other embodiments the at least one port comprises a plurality of ports formed in the wear layer.

In some embodiments the ultrasound device includes a plurality of transducer elements and at least one port is formed between the transducer elements.

The ultrasound device may optionally include a degassing unit in fluid communication with the lubricant reservoir.

The ultrasound device may include two or more lubricant reservoir and may include a valve interposed between the pump and the two or more reservoir for selecting a desired lubricant reservoir.

### BRIEF DESCRIPTION OF THE DRAWINGS

FIG. 1 is a schematic diagram of the present invention.

### DETAILED DESCRIPTION OF THE PREFERRED EMBODIMENTS

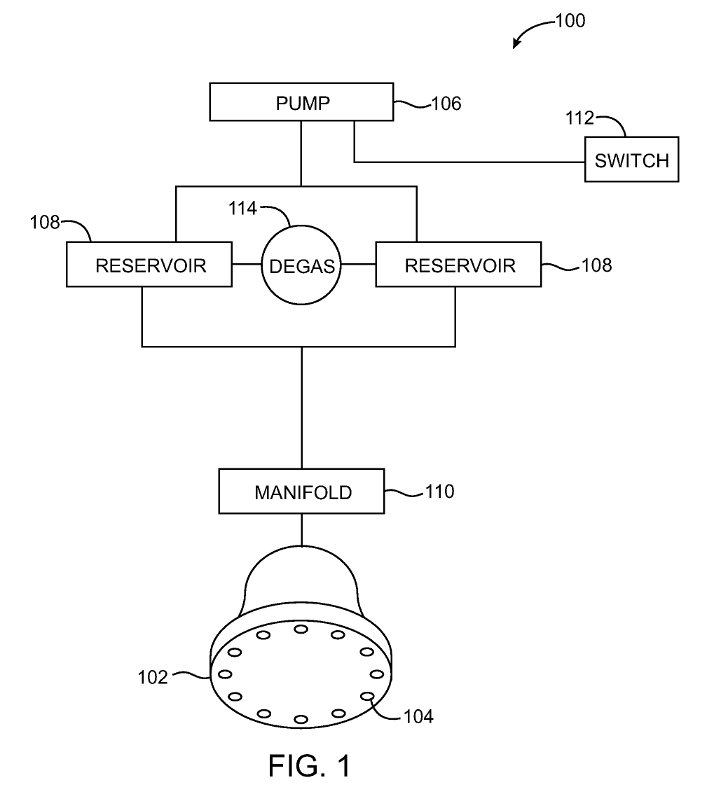

FIG. 1 is a schematic diagram of an ultrasound device 100 according to the present invention. The ultrasound device 100 includes a housing (not shown), an ultrasound transducer 102 at least partially housed within the housing. The ultrasound transducer 102 having a wear layer, i.e., a surface configured for acoustic coupling to a treatment area.

At least one and preferably two or more lubricant dispenser ports 104 are provided on the wear surface (wear layer) of the transducer 102 or on the housing in a location proximate the wear surface of the transducer 102. For example, the dispenser ports 104 may be formed in the housing along the periphery of the wear surface.

The ultrasound device may include two or more transducers 102 and the dispenser ports 104 may be dispersed between the transducers 102.

The lubricant dispenser ports 104 are in fluid communication with a pump 106 and one or more lubricant reservoir 108. If plural lubricant dispenser ports 104 are provided, the ports 104 may be connected to an optional manifold 110 which distributes the lubricant pumped from the reservoir 108 among the ports 104. As noted, the device 100 may include two or more reservoirs 108 and a valve (not shown) enabling the operator to select the desired reservoir 108 from which the lubricant is dispensed. Each reservoir 108 may, for example, contain a different lubricant thereby enabling the operator to switch between lubricants without the need to empty and refill the reservoir 108. The use of different lubricants may, for example, be indicated for different parts of the body. The valve 116 may be electronically actuated or may be manually selectable.

The pump 106 is preferably actuated by a switch 112 which does not require the operator to interrupt the general flow of the ultrasound treatment. Thus the switch 112 may be foot actuated or may be provided on the ultrasound device 100 in an easily accessible location. The switch 112 may be a simple on/off switch, or may have additional settings such as low, medium and high such that the lubricant is dispensed from the ports 104 in various volumes depending on the setting of switch 112. The valve (not shown) may be operably connected to the switch 112 so that the operator may additionally select the desired lubricant to be dispensed.

According to one aspect of the invention a degassing mechanism 114 such as is commonly known in the art is provided in fluid communication with the lubricant reservoir 108. The degassing mechanism 114 degasses the lubricant thereby eliminating gas which could react with and/or degrade the ultrasound transmission to the treatment area. Alternatively, the lubricant may be degassed prior to storage in the reservoir 108.

The dispenser ports 104 may be positioned in any of a variety of layouts. The ideal number and arrangement of the ports 104 varies depending on the viscosity of the lubricant to be used. The device 100 shown in FIG. 1 includes ten ports 104; however, the device may include one or more ports 104.

The invention may be embodied in other forms without departure from the spirit and essential characteristics thereof. The embodiments described therefore are to be considered in all respects as illustrative and not restrictive. Although the present invention has been described in terms of certain preferred embodiments, other embodiments that are apparent to those of ordinary skill in the art are also within the scope of the invention. Accordingly, the scope of the invention is intended to be defined only by reference to the appended claims.

I claim:

1. An ultrasound device, comprising:
a housing;
an ultrasound transducer disposed within the housing and having a wear layer;
at least one lubricant dispensing port formed on the housing;
a lubricant reservoir operably connected to the at least one lubricant dispensing port;
a pump in fluid connection with the lubricant reservoir, the pump dispensing lubricant from the reservoir through the at least one dispensing port; and
a pump actuator operably connected to the pump and controlling the pump.

2. The ultrasound device according to claim 1, further comprising a manifold in fluid connection with the pump, and the manifold distributes lubricant among the at least one dispensing port.

3. The ultrasound device according to claim 1, wherein the pump actuator is a foot switch for controlling operation of the pump.

4. The ultrasound device according to claim 1, wherein the pump actuator is a switch mounted on the housing for controlling operation of the pump.

5. The ultrasound device according to claim 1, wherein the at least one port is formed along a periphery of the wear layer.

6. The ultrasound device according to claim 1, wherein the at least one dispensing port comprises at least two dispensing ports.

7. The ultrasound device according to claim 1, wherein the at least one dispensing port is formed in the wear layer.

8. The ultrasound device according to claim 1, wherein the ultrasound transducer includes a plurality of transducer elements and at least one port is formed between the transducer elements.

9. The ultrasound device according to claim 1, further comprising a degassing unit in fluid communication with the lubricant reservoir.

10. The ultrasound device according to claim 1, further comprising a degassing means in fluid communication with the lubricant reservoir.

11. The ultrasound device according to claim 1, wherein
the lubricant reservoir includes at least two lubricant reservoirs; and
a valve is interposed between the pump and the at least two reservoir for selecting a desired one of the at least two lubricant reservoir.

12. The ultrasound device according to claim 11, wherein the valve is electronically controlled by the pump actuator.
